(19) United States
(12) Patent Application Publication
(10) Pub. No.: US 2010/0179352 A1
(43) Pub. Date: Jul. 15, 2010

(54) PROCESS FOR THE PREPARATION OF CYCLOPENTANONE

(75) Inventors: Joaquim Henrique Teles, Otterstadt (DE); Mathieu Chabanas, Berg (DE); Thomas Genger, Lambsheim (DE); Beatrice Rossler, Weisenheim Am Sand (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE Patents, Trademarks and Licences, Ludwigshafen (DE)

(21) Appl. No.: 12/602,836

(22) PCT Filed: May 27, 2008
(86) PCT No.: PCT/EP2008/056445

§ 371 Date: Dec. 3, 2009

(30) Foreign Application Priority Data
Jun. 4, 2007 (EP) 07109494.0

(51) Int. Cl. C07C 45/28

(52) U.S. Cl. 568/379

(57) ABSTRACT

The invention relates to a method for producing cyclopentanone. Said method comprises the step of reacting a mixture (G1), which contains at least cyclopentene, with a mixture (G2), which contains at least dinitrogen monoxide. The reaction is carried out in at least one reactor (R1) having channels with a diameter in the range of 0.1 mm to 50 mm, the reactor comprising at least two zones (Z1) and (Z2) having channels with different diameters and the diameters of the channels of zone (Z1) being smaller than the diameter of the channels of zone (Z2).

The present invention relates to a process for the preparation of cyclopentanone comprising the reaction of a mixture (G1) containing at least cyclopentene with a mixture (G2) containing at least dinitrogen monoxide wherein the reaction is executed in at least one reactor (R1) having channels of a diameter in the range of from 0.1 mm to 50 mm wherein the reactor has at least two zones (Z1) and (Z2) having channels of different diameters, and the diameter of the channels of zone (Z1) is smaller than the diameter of the channels of zone (Z2).

Processes for the preparation of cyclopentanone are basically known in the prior art. Likewise, it is known that cyclopentanone can be obtained by the reaction of cyclopentene with dinitrogen monoxide. The preparation of cyclopentanone by the oxidation of cyclopentene with N₂O is a very selective reaction which is strongly exothermic.

For instance, GB 649,680 discloses the reaction of alkenes, like for example cyclohexene or cyclopentene with N₂O. However, the reaction of cyclopentene with N₂O is not explicitly described in the examples of these documents. Other substituted or unsubstituted olefins which are reacted in the examples are used either as pure compounds or together with the solvent dimethylanilin. U.S. Pat. No. 2,636,898 being equivalent to GB 649,680 does not disclose the reaction of cyclopentene with N₂O in the examples either. In these examples as well, the un-substituted olefins are reacted with N₂O exclusively in pure form without the addition of a solvent. The reaction occurs at 300° C. and 500 atm.

In J. Chem. Soc., pp. 2999-3008 (1951) F. S. Bridson-Jones et al. describe the reaction of olefins with N₂O, wherein for example cyclohexene is reacted to cyclohexanone. Also in this case cyclohexene is added as such without the addition of an additional solvent, for instance. Likewise the reaction of ethylene, acenaphtylene and methylene cyclobutane is described, wherein either cyclohexane or decaline is used as solvent.

Also K. A. Dubkov et al., React. Kinet. Catal. Lett., Vol. 77, No. 1, pp. 197-205 (2002) describe the reaction of pure cyclopentene of 99 percent with pure N₂O (pure, medical grade).

In recent scientific articles of G. L. Panov et al., “Non-catalytic Liquid Phase Oxidation of Alkenes with Nitrous Oxide. 1. Oxidation of Cyclohexene to Cyclohexanone”, React. Kinet. Catal. Lett. Vol. 76, No. 2 (2002) pp. 401-405, and K. A. Dubkov et al., “Non-catalytic Liquid Phase Oxidation of Alkenes with Nitrous Oxide. 2. Oxidation of Cyclopentene to Cyclopentanone”, React. Kinet. Catal. Lett. Vol. 77, No. 1 (2002) pp. 197-205 oxidations of olefinic compounds with dinitrogen monoxide are also described. Also a scientific article “Liquid Phase Oxidation of Alkenes with Nitrous Oxide to Carbonyl Compounds” by E. V. Starakon et al. in Adv. Synth. Catal. 2004, 346, 268-274 contains a mechanistic study of the oxidation of alkenes with dinitrogen monoxide in liquid phase.

The synthesis of carbonyl compounds from alkenes with dinitrogen monoxide is also described in different international patent applications. For instance, the WO 03/078370 discloses a process for the preparation of carbonyl compounds from aliphatic alkenes with dinitrogen monoxide. The reaction is carried out at temperatures in the range of from 20 to 350° C. and pressures from 0.01 to 100 atm. The WO 03/078374 discloses a corresponding process for the preparation of cyclohexanone. According to WO 03/078372 cyclic ketones with 4 to 5 C atoms are produced. According to WO 03/078375 under these process conditions cyclic ketones are prepared from cyclic alkenes having 7 to 20 C atoms. WO 03/078371 discloses a process for the preparation of substituted ketones from substituted alkenes. WO 04/000777 discloses a process for the reaction of di- and polyalkenes with dinitrogen monoxide to the corresponding carbonyl compounds.

The DE 103 19 489.4 discloses a process for the preparation of cyclopetanone using dinitrogen monoxide as oxidizing agent.

WO 2006/032532 discloses a process for the preparation of cyclopentanone, starting from a mixture containing cyclopentene to 95% by weight at the most. Besides cyclopentene the used mixture can comprise further solvents, wherein for example further hydrocarbons are mentioned as components of the mixture. In that case cyclopentanone is obtained by reaction with N₂O, wherein either pure N₂O or a gas mixture comprising N₂O is used in liquid or supercritical form. Preferably, the process disclosed therein is carried out in a batch reactor.

Since the reaction is of first order each with respect to cyclopentene and N₂O the reaction rate drops quickly with the conversion which complicates achieving high conversions of both reactants.

The reaction is strongly exothermic, such that even in the plate heat exchangers known as efficient heat exchangers the heat removal is not sufficient to avoid temperature peaks (so called “hot spots”) at a singular injection. This is even more the case for shell-and-tube heat exchangers.

Because of the high reaction temperature and the reaction rate being in first assumption proportional to the second power of the density and to the product of the concentration of the reactants, the reaction is carried out at increased pressure and with a concentration of educts as high as possible in order to keep the reaction volume as small as possible. Since the reaction proceeds fast under these conditions and is strongly exothermic a (pressure-resistant) reactor is required that allows high rates of heat removal.

A dilution of reactants does lead to more easily manageable rates of heat production but in that case consequently also to a much higher reactor volume and thus, to significantly bigger production plants and high costs associated therewith because of the significantly higher substance flows that have to be moved.

Basically, it is known that in particular for exothermic reactions the reaction can be carried out in micro reactors.

Micro structured reactors or heat exchangers having a microstructure are basically known in prior art. For example, DD 246257 A1 already describes micro apparatuses and processes for their production. The micro apparatuses disclosed therein are produced from stacks of substrate lamellas, wherein the hollows incorporated into the single substrate lamellas vary in their shape and surface design.

WO 94/21372 also discloses reactors that are built up of different layers and that contain micro structured channels. Such devices can be used for chemical reactions.

WO 01/54806 discloses a reactor with heat exchanger. In that case the heat exchanger is composed of a multitude of metal plates lying upon each other each of them containing channels in which the heat exchanging agent can flow.

Also EP 0 212 878 A1 discloses heat exchangers made up of single plates wherein the single plates each contain micro structured channels. According to EP 0 212 878 A1 in that case the channels have a radius in the range of from 0.2 to 1.5 mm.

WO 03/055585 discloses a chemical reactor. This reactor is made up of a stack of interconnected metal plates each of them having hollows so that reaction channels are formed.

WO 01/54805 relates to reactors having microstructures that are present in spiral form in the reactor. Also WO 01/54804 discloses heat exchangers that are made up of single plates having microstructures, where it is also disclosed that the heat exchangers disclosed therein can also be used as chemical reactors.

In WO 2004/099696 heat exchangers built up of plates are disclosed. In that case the individual plates initially have branching zones, micro structured zones and again a zone in which the single flows are merged.

In DE 10036602 A1 a micro reactor for reactions of gases with fluids is disclosed. The micro reactors disclosed therein are suitable for carrying out chemical reactions of a reaction partner in fluid form with a reaction partner in gaseous form, optionally in the presence of a solid catalyst. The chemical process control occurs in areas which are built up of two or more, substantially plane-parallel plates or layers.

Also known in the art are chemical processes that can be carried out in such micro structured reactors. In this way EP 1 586 372 A1 discloses an alkoxylation in micro structured capillary reactors. A process and a device for the preparation of polyether alcohols by alkoxylation of alcohols are disclosed therein. The process is executed in a micro structured reactor in the fluid phase. The single channels of the reactor having example a diameter of less than 2 mm are cooled by means of a cooling medium or heated by means of a heating medium.

Based on the state of the art an object of the present invention was to provide a process for the preparation of cyclopentanone in which the educts can be reacted with high space-time yield and good conversions.

A further object of the present invention was to provide a process for the preparation of cyclopentanone in which the educts can be reacted with high space-time yield and good conversions at the same time minimizing the reactor costs.

A further object of the present invention was to provide a process for the preparation of cyclopentanone in which the educts can be reacted with high space-time yield and with good conversions and which ensures a safe process control.

According to the invention, this object is solved by a process for the preparation of cyclopentanone comprising the reaction of a mixture (G1) containing at least cyclopentene with a mixture (G2) containing at least dinitrogen monoxide, wherein the reaction is carried out in at least one reactor (R1) having channels of a diameter in the range of from 0.1 to 50 mm, wherein the reactor has at least two zones (Z1) and (Z2) having channels of a different diameter, and the diameter of the channels of zone (Z1) is smaller than the diameter of the channels of zone (Z2).

According to the invention, the mixture (G1) contains at least cyclopentene. According to the invention, the mixture (G2) contains at least dinitrogen monoxide. According to the process of the invention, the mixtures (G1) and (G2) are reacted with each other in gaseous or liquid or supercritical form. Preferably the mixtures (G1) and (G2) are reacted with each other in supercritical form according to the invention.

According to the invention, the reactor (R1) having channels of a diameter in the range of from 0.1 mm to 50 mm can be used alone or as main reactor in combination with other reactors. In that case it is possible that the reactor (R1) is combined with a conventional reactor, for example a tubular reactor or a shell-and-tube reactor or a further reactor having channels of, particularly, a diameter in the range of from 0.1 mm to 50 mm. According to the invention, it is in that case possible to operate the different reactors in serial or parallel manner or a combination of both.

According to the invention, the reactor (R1) has channels of a diameter in the range of from 0.1 to 50 mm. In that case the process according to the invention is executed in such a way that the reaction of mixtures (G1) and (G2) occurs in the channels. According to the invention, the channels constitute the reaction volume of reactor (R1).

The diameter of the channels through which the reactants flow can be substantially identical within one zone and is in the range of from 0.1 mm to 50 mm. In a preferred embodiment of the process the channels are semicircular and have a radius between 0.05 and 25 mm.

According to the invention, the diameter of the channels within one zone is substantially identical. In that case the single channels can be arranged parallel or they can have other geometric arrangements.

The reactor (R1) can for example consist of a stack of diffusion welded metal plates in which channels are incorporated by means of an appropriate method. Basically, this type of construction is known in the state of the art, for example in EP 0212878 A1 or WO 2004/099696.

The reactor (R1) can for example also have a device for heat exchange. A micro reactor built up of plates can for example have layers in which a heating or cooling medium flows.

The manufacture of the channels can be made for example in a multistage process, wherein in a first step the capillary structure in form of channel crowds or groove crowds in single plates is generated by for example milling, etching, stamping or similar processes and subsequently a connection of the plates is effected for example by diffusion welding or soldering. Every single channel is thus isolated from adjacent channels. Different zones with channels of different diameters can in principle be generated by combining plates with channels of different diameters.

The mixing of the mixtures (G1) and (G2) can occur inside as well as outside of the reactor. In the case of mixing outside of the reactor an appropriate mixing unit is located upstream of the reactor. Preferably, mixing of the educts however occurs in the reactor, preferably, in the channels.

In that case, according to the invention, it is particularly preferred to guide the mixture (G1) within the plate in channels that branch and thereby narrow to a diameter from 0.1 mm to 50 mm, and to guide the mixture (G2) on the same plate or a different plate in separate channels, that likewise branch and narrow to a diameter from 0.1 mm to 50 mm before the mixtures (G1) and (G2) are brought into contact with each other. For contacting the mixtures (G1) and (G2) for example the streams from two in each case adjacent plates can be merged. For this purpose openings can be in all channels of the one plate, through which then the streams from the channels of the other adjacent plate are guided.

So, the flows each from one channel for mixture (G1) and one channel for mixture (G2) are merged.

According to the invention, the reactor (R1) can have an entry zone. Such an entry zone consists for example of at least one plate and carries the two separated entries for mixtures (G1) and (G2). The two entry channels are initially branched and the mixtures (G1) and (G2) are only mixed with each other when the channels have a cross sectional area which is comparable to the cross sectional area of the channels in the zone (Z1).

At the mixing positions the mixtures (G1) and (G2) are guided for example through perpendicular drill holes into the first product plate of the reaction zone. In a possible embodiment of the entry zone the channels for the mixture (G1) and the mixture (G2) are divided into different plates.

For example, mixture (G1) is divided in the first plate and is guided through perpendicular channels into the next plate where it merges with divided mixture (G2) to form a mixed feed that can again be guided through perpendicular channels into a further plate. According to the invention it is also possible that there are further plates having channels for a cooling medium between the single plates.

Based on the reaction conduct according to the invention the process according to the invention is particularly advantageous concerning safeguard technology because a particularly intensive cooling is possible by means of the reaction conduct in reactor (R1).

In principle, a merging of the channels to broader channels can occur at the reactor exit. The channels of the last product plate can for example be merged to an exit line. According to the invention it is also possible to replace this exit zone by a hood, for example, if the conversion of cyclopentene and dinitrogen monoxide is at least 90%.

Therefore, according to a further embodiment the present invention also relates to a process for the preparation of cyclopentanone comprising the reaction of a mixture (G1) containing at least cyclopentene with a mixture (G2) containing at least dinitrogen monoxide in at least one reactor (R1) having channels of a diameter in the range of from 0.1 mm to 50 mm, wherein the mixtures (G1) and (G2) are mixed in the reactor in the channels.

According to the invention, merging of the mixtures (G1) and (G2) can for example occur as such that the reaction mixture enters directly behind the mixing positions in a wall-cooled area of the plate, in which the famously advantageous intensive heat transfer condition in micro structures can be used and a reaction conduct with negligible superelevations of temperature is possible. The cooling channels in the cooling plates can be arranged in a parallel or perpendicular manner towards the direction of flow of the reaction medium. Thus, a cocurrent flow, a counter-current flow and a cross flow operation of the cooling medium is possible.

Thus, the process according to the invention allows to limit or to reduce, respectively, hot spots, i.e., local overheatings. According to the invention, it is especially preferred to conduct the process such that each at the beginning of a zone a hot spot occurs in a permitted area, wherein for example an increase of temperature in the range of 10° C. occurs. At the same time, according to the invention, the temperature in the reactor at a IS distinct point in the reactor is 350° C. at the most, preferably 340° C. at the most, particularly 320° C. at the most, more preferably 310° C. at the most, particularly preferably 300° C. at the most.

A further advantage of the process, according to the invention, is that an enlargement of the scale (scale-up) can be for example simply performed by modular conduct of the number of the channels in micro structured reactors or by increasing the numbers of micro structured reactors. Thus, the production capacity can be increased simple and without a risk.

Thus the micro structured reactor (R1) used according to the invention can be particularly designed for the execution of continuous processes in the fluid phase.

The reactor (R1) used according to the invention has at least two zones (Z1) and (Z2) with channels of different diameters and the diameter of the channels of zone (Z1) is smaller than the diameter of the channels of zone (Z2). Accordingly, the diameter of the channels increases from zone (Z1) to zone (Z2), wherein according to the invention it is possible to adjust the reaction conditions in such a way that each at the beginning of a zone a hot spot occurs in a permitted area. Thus, according to the invention it is possible to conduct the reaction course over reactor (R1) in such a way that the reaction volume is utilized optimally. In comparison to a reactor having channels of essentially constant diameter the reactor volume in case of reactor (R1) used according to the invention is better utilized by comparatively more reactor volume by means of increasing diameter of the channels.

The reactor (R1) used according to the invention has at least two zones (Z1) and (Z2) having channels of a diameter in the range of from 0.1 mm to 50 mm. Moreover, the reactor (R1) can have further zones, for example further zones with channels of a diameter in the range of from 0.1 mm to 50 mm or zones in which the mixtures (G1) or (G2) or (G1) and (G2) are introduced into the reactor and which have channels that branch and their diameter narrows to a diameter in the range of the diameters of the channels in zones (Z1) and (Z2).

According to the invention, the reactor (R1) can for example also have a zone in which mixtures (G1) and (G2) are brought into contact or a zone, for example an exit zone, in which the channels are merged and get wider.

The process according to the invention inter alia has the advantage that the reaction of cyclopentene and dinitrogen monoxide with high space-time yield can be carried out safety-related unobjectionably. The process further allows to design the micro reactor preferably cost saving because by means of zones having channels of different diameters the reaction conduct can be designed such that each at the beginning of a zone an increase of temperature can again occur, wherein the maximum temperature at a distinct point within the reactor according to the invention is not exceeded. By means of highly efficient heat removal it can be operated without diluting additives.

The process according to the invention allows a reaction of mixture (G1) with mixture (G2) with a conversion based on dinitrogen monoxide of more than or equal to 80%, preferably of more than or equal to 85%, particularly preferably of more than or equal to 95%. At the same time according to the invention the conversion based on cyclopentene is more than or equal to 50%, in particular more than or equal to 55%, particularly preferably more than or equal to 60%.

The upper limit of the conversions based on both cyclopentene and on dinitrogen monoxide is in general at 90%, preferably at 92%, particularly at 95%, more preferably at 98% and particularly preferably at 99%.

The molar ratio between N₂O in mixture (G2) and cyclopentene in mixture (G1) is adjusted according to the invention in such a way that the ratio is greater than 0.5, preferably greater than 0.6.

The reactor (R1) used according to the invention has at least two zones (Z1) and (Z2) having channels of a diameter in the range of from 0.1 mm to 50 mm. According to the invention the reactor (R1) can have even more zones having channels of a diameter in the range of from 0.1 mm to 50 mm, for example 3, 4 or 5 zones, particularly preferably 3 zones (Z1), (Z2) and (Z3), wherein the zones each have channels of a diameter in the range of from 0.1 mm to 50 mm. According to the invention the diameter of the channels each is smaller in one zone than in the following zone.

According to the invention, the part of the total reaction volume built up of the channels of the first zone (Z1) in relation to the total reaction volume within the reactor is for example 1 to 60%, particularly 5 to 55%, more preferably 10 to 50%, particularly preferably 15 to 45%, for example 20%, 25%, 30%, 35% or 40%.

Therefore, according to a further embodiment, the present invention also relates to a process for the preparation of cyclopentanone comprising the reaction of a mixture (G1) containing at least cyclopentene with a mixture (G2) containing at least dinitrogen monoxide in at least one reactor (R1) having channels of a diameter in the range of from 0.1 mm to 50 mm, wherein the reactor (R1) has at least three zones (Z1), (Z2) and (Z3) having channels of a diameter in the range of from 0.1 mm to 50 mm, and the diameter of the channels of zone (Z1) is smaller than the diameter of the channels of zone (Z2) and the diameter of channels of zone (Z2) is smaller than the diameter of channels of zone (Z3).

According to the invention, the diameter of the channels in zones (Z1), (Z2) and (Z3) is in the range of from 0.1 mm to 50 mm.

For example, the diameter of the channels in zone (Z1) is in the range of from 0.5 mm to 2 mm, preferably in the range of from 0.7 mm to 1.8 mm, particularly in the range of from 0.9 mm to 1.5 mm. For example, the diameter of the channels of zone (Z2) is in the range of from 2.5 mm to 6 mm, preferably in the range of from 3 mm to 5.5 mm, particularly in the range of from 3.5 mm to 5 mm. For example the diameter of the channels in zone (Z3) is in the range of from 6.5 mm to 10 mm, preferably in the range of from 7 mm to 9.5 mm, particularly in the range of from 7.5 mm to 9 mm.

Therefore, according to a further embodiment, the present invention also relates to a process for the preparation of cyclopentanone comprising the reaction of a mixture (G1) containing at least cyclopentene with a mixture (G2) containing at least dinitrogen monoxide in at least one reactor (R1) having channels of a diameter in the range of from 0.1 mm to 50 mm, wherein the diameter of the channels in zone (Z1) is in the range of from 0.5 mm to 2.0 mm, the diameter of the channels in zone (Z2) is in the range of from 2.5 mm to 6.0 mm and the diameter of the channels in zone (Z3) is in the range of from 6.5 mm to 10.0 mm.

According to the invention, also several reactors can be used for the process, for example two reactors (R1) and (R2) having channels of a diameter in the range of from 0.1 mm to 50 mm, particularly those which have zones having channels of a different diameter. In that case the reactors (R1) and (R2) used can have the same or a different number of zones. For example reactor (R1) can have two zones (Z1) and (Z2) and the reactor (R2) three zones (Z1), (Z2) and (Z3). Preferably, both reactors (R1) and (R2) have three zones (Z1), (Z2) and (Z3), wherein the diameter of the channels in both reactors can vary in the range according to the invention.

Therefore, according to a further embodiment the present invention also relates to a process for the preparation of cyclopentanone comprising the reaction of a mixture (G1) containing at least cyclopentene with a mixture (G2) containing at least dinitrogen monoxide in at least one reactor (R1) having channels of a diameter in the range of from 0.1 mm to 50 mm, wherein the reaction is carried out in two parallel reactors (R1) and

(R2) each having channels of a diameter in the range of from 0.1 mm to 50 mm, and, wherein reactor (R1) and reactor (R2) each have at least two zones (Z1) and (Z2) having channels of a different diameter and the diameter of the channels of zone (Z1) each is smaller than the diameter of the channels of zone (Z2).

If several reactors (R1) and (R2) are used these can be according to the invention connected in parallel or in series, preferably in parallel. According to the invention it is also possible to connect two reactors (R1) and (R2) having channels of a diameter in the range of from 0.1 mm to 50 mm in parallel and to install a further reactor downstream, for example a tube reactor or a shell-and-tube reactor as a downstream reactor.

The reaction conditions for the process, according to the invention, can be varied in wide ranges. Accordingly, the reaction is preferably carried out at a temperature in the range of from 200 to 350° C., preferably at 230 to 340° C., particularly at 250 to 320° C., particularly preferably at 270 to 300° C., for example at 280° C., 285° C., 290° C. or 295° C.

Preferably, the reaction is carried out at pressures from 200 to 500 bar, preferably at 220 to 450 bar, particularly at 240 to 400 bar, particularly preferably at 260 to 350 bar, for example at 265 bar, 270 bar, 275 bar, 280 bar, 285 bar, 290 bar, 295 bar, 300 bar, 305 bar, 310 bar, 315 bar, 320 bar, 325 bar, 330 bar, 335 bar, 340 bar or 345 bar.

According to a preferred embodiment, the reaction is carried out at a temperature range between 270 and 300° C. and at a pressure from 260 to 350 bar, particularly 280 bar.

The reaction conditions are preferably selected such that the conversion of N₂O is over to 80% and the conversion of cyclopentene is over 50%.

Therefore, according to a further embodiment the present invention also relates to a process for the preparation of cyclopentanone comprising the reaction of a mixture (G1) containing at least cyclopentene with a mixture (G2) containing at least dinitrogen Is monoxide in at least one reactor (R1) having channels of a diameter in the range of from 0.1 mm to 50 mm, wherein the reaction is carried out at a pressure from 200 to 500 bar and a temperature from 270 to 300° C.

According to the invention, the mixture (G1) contains at least cyclopentene. In that case the mixture (G1) contains preferably at least 90% by weight of cyclopentene, for example 90 to 99% by weight of cyclopentene, preferably 91 to 95% by weight of cyclopentene, particularly 92% by weight, 93% by weight or 94% by weight of cyclopentene.

Therefore, according to a further embodiment the present invention also relates to a process for the preparation of cyclopentanone comprising the reaction of a mixture (G1) containing at least cyclopentene with a mixture (G2) containing at least dinitrogen monoxide in at least one reactor (R1) having channels of a diameter in the range of from 0.1 mm to 50 mm, wherein the mixture (G1) contains at least 90% by weight of cyclopentene.

In addition to cyclopentene, the mixture (G1) can in principle also contain every further compound. Suitable are inter alia also compounds that can likewise react with N₂O. Preferred are herein such compounds that indeed can in principle react with N₂O yet are inert towards N₂O under the reaction conditions chosen according to the invention. The term “inert” as used in the context of the present invention denotes compounds that under the reaction conditions chosen according to the invention either do not react with N₂O or in comparison to the reaction of cyclopentene with N₂O react in such a limited way that their reaction product with N₂O is contained in the resulting mixture to 5% by weight at the most, preferably to 3% by weight at the most and particularly preferably to 2% by weight at the most, each based on the total weight of the resulting mixture.

The content of by-products in mixture (G1) is for example smaller than 15% by weight, preferentially smaller than 12% by weight, preferably smaller than 10% by weight, particularly smaller than 8% by weight, particularly preferably smaller than 5% by weight.

According to a further preferred embodiment of the present invention the mixture (G1) consists of at least 99% by weight of hydrocarbons based on the total weight of the mixture (G1). Besides the hydrocarbons the mixture (G1) can accordingly contain another 1% by weight of at least one further compound wherein inter alia at least one of the above mentioned preferably inert compounds different from hydrocarbons can be contained to 1% by weight at the most. Other compounds can be also encompassed up to 1% by weight at the most with the provision that they do not interfere with the reaction of cyclopentene with mixture (G2).

The term “hydrocarbons” as used in the context of the present invention denotes compounds of which each is a non substituted hydrocarbon and therefore does only consist of the atoms C and H. More preferably, the mixture contains further compounds to 0.5% by weight at the most, more preferably to 0.1% by weight at the most, more preferably to 0.01% by weight at the most and particularly preferably to 0.001% by weight at the most. Particularly preferred are mixtures (G1) which contain no further compounds besides hydrocarbons within the limits of measuring accuracy of the respective analytical methods used.

According to a preferred embodiment, the mixture (G1) is gaseous, fluid or supercritical, preferably supercritical at the reaction conditions chosen according to the invention.

Within the scope of a likewise preferred embodiment of the process according to the invention a mixture (G1) is used that consists of at least 90% by weight, preferably of at least 95% by weight, particularly of at least 99% by weight of C₅-hydrocarbons and hydrocarbons with more than 5 carbon atoms. Besides cyclopentene at least a further C₅-hydrocarbon or at least a hydrocarbon with more than 5 carbon atoms or a mixture of at least a further C₅-hydrocarbon and at least a hydrocarbon with at least more than 5 carbon atoms can be contained in (G1).

Accordingly, the present invention also describes a process as described above characterized in that the mixture (G1) contains at least 99% by weight of C₅-hydrocarbons and hydrocarbons with more than 5 carbon atoms.

The corresponding hydrocarbons already discussed above in context with inert compounds are used amongst others as particularly preferred hydrocarbons with more than 5 carbon atoms.

According to the invention, preferably as mixture (G1) such mixtures are used that arise in large-scale processes. Within the scope of the present invention mixtures are preferred herein that consist of at least 95% by weight, more preferably of at least 97% by weight and particularly preferably of at least 99% by weight of C₅-, C₆- and C₇-hydrocarbons.

Accordingly, the present invention also relates to a process as described above that is characterized in that the mixture (G1) contains at least 99% by weight of C₅- and C₆- or C₅- and C₇- or C₅- and C₆- and C₇-hydrocarbons.

Within the scope of the present invention, in that case the mixture (G1) can besides cyclopentene contain either at least a further C₅-hydrocarbon or at least a C₆-hydrocarbon or at least a C₇-hydrocarbon or a mixture of at least a further C₅-hydrocarbon and at least a C₆-hydrocarbon or a mixture of at least a further C₅-hydrocarbon and at least a C₇-hydrocarbon or a mixture of at least a further C₅-hydrocarbon and at least a C₆-hydrocarbon and at least a C₇-hydrocarbon.

Within the scope of a preferred embodiment of the process according to the invention a hydrocarbon mixture is used as a mixture (G1) that is obtained from a steam cracker or a refinery and that contains cyclopentene. In this context, for example C₅-cuts from steam crackers are preferred that substantially contain only C₅- and C₆-hydrocarbons. Hydrocarbons with more than 6 carbon atoms are usually not contained in industrially obtained C₅-cuts that besides cyclopentene comprise for example 2-butene, isopentane, 1-pentene, 2-methylbutene-1, trans-2-pentene, n-pentane, cis-2-pentene, 2-methylbutene-2, cyclopentane, 2,2-dimethylbutane, 2-methylpentane, 3-methylpentane, n-hexane and benzene. In general the C₅-cut from a steam cracker contains cyclopentene in the range of from 5 to 60% by weight and preferably in the range of from 15 to 50% by weight. Such mixtures are preferably further purified before being used as mixtures (G1) in the process according to the invention.

Therefore, the present invention also relates to a process as described above that is characterized in that the mixture (G1) contains at least 99% by weight of a mixture of C₅- and C₆-hydrocarbons.

According to the invention, this mixture of substantially C₅- and C₆-hydrocarbons that is preferably obtained as C₅-cut from a steam cracker can be used as such. Prior to the reaction according to the invention the mixture of substantially C₅- and C₆-hydrocarbons is preferably subjected to purification in which preferably compounds having a lower boiling point in comparison to cyclopentene are separated off. While all conceivable methods can be used herein the distillative separation of the mixture is preferred.

Within the scope of the present invention mixtures (G1) are preferably obtained herein that contain at the most 10% by weight of C₅- and/or C₆-hydrocarbons that boil at lower temperatures than cyclopentene. If optionally at least one C₄-hydrocarbon is additionally contained in the mixture that is to be purified, then preferably mixtures (G1) are obtained by means of the preferably used distillation that contain 25% by weight at the most of C₄- and/or C₅- and/or C₆-hydrocarbons boiling at lower temperatures than cyclopentene. Within the scope of the present invention mixtures (G1) are preferably obtained in doing so that contain 15% by weight at the most, more preferably 10% by weight at the most and particularly preferably 5% by weight at the most of C₅- and/or C₆-hydrocarbons boiling at lower temperatures than cyclopentene. If optionally at least one C₄-hydrocarbon is additionally contained in the mixture that is to be purified, then preferably mixtures (G1) are obtained by means of the preferably used distillation that contain preferably 5% by weight at the most, more preferably 3% by weight at the most and particularly preferably 2% by weight at the most of C₄- and/or C₅- and/or C₆-hydrocarbons boiling at lower temperature than cyclopentene.

The mixtures obtained in such a way generally contain cyclopentene in a range of from 80 to 99.99% by weight, preferably in a range of from 85 to 99% by weight and particularly preferably in a range of from 90 to 95% by weight. Such mixtures can be further purified or concentrated before they can be used as mixtures (G1) according to the invention.

In particular the present invention also relates to the use of a cyclopentene containing mixture of hydrocarbons as educt for the preparation of cyclopentanone characterized in that the hydrocarbon mixture containing cyclopentene is either the C₅-cut of a steam cracker plant or the mixture obtained from the partial hydrogenation of cyclopentadien containing cyclopentene or a mixture of the C₅-cut of a steam cracker plant and the mixture obtained from the partial hydrogenation of cyclopentadien containing cyclopentene.

According to the invention, the mixture (G2) contains at least 70% by volume of dinitrogen monoxide, for example 70 to 100% by volume. Preferably, the mixture (G2) contains at least 75% by volume of dinitrogen monoxide, particularly at least 80% by volume, preferably at least 85% by volume. The mixture (G2) contains preferably 75 to 99% by volume of dinitrogen monoxide, more preferably 80 to 95% by volume, particularly preferably 82 to 90% by volume, for example 83% by volume, 84% by volume, 85% by volume, 86% by volume, 87% by volume, 88% by volume or 89% by volume.

Therefore, according to a further embodiment the present invention also relates to a process for the preparation of cyclopentanone comprising the reaction of a mixture (G1) containing at least cyclopentene with a mixture (G2) containing at least dinitrogen is monoxide in at least one reactor (R1) having channels of a diameter in the range of from 0.1 mm to 50 mm, wherein the mixture (G2) contains at least 70% by volume of dinitrogen monoxide.

According to a further embodiment, the present invention also relates to a process for the preparation of cyclopentanone comprising the reaction of a mixture (G1) containing at least cyclopentene with a mixture (G2) containing at least dinitrogen monoxide in at least one reactor (R1) having channels of a diameter in the range of from 0.1 mm to 50 mm, wherein the mixture (G2) contains 75 to 99% by volume of dinitrogen monoxide.

In principle the mixture (G2) containing dinitrogen monoxide can be obtained from any source.

According to the invention, this mixture (G2) is preferably liquefied and then used in liquid form. In that case dinitrogen monoxide or the gas mixture containing dinitrogen monoxide may be liquefied by all processes known to the skilled person, in particular by suitable selection of pressure and temperature.

According to the invention, the mixture (G2) can also contain at least another gas besides N₂O. Herein substantially all gases are conceivable, as long as it is provided that the reaction according to the invention of cyclopentene with N₂O is possible. In particular mixtures (G2) are therefore preferred that besides N₂O contain at least one inert gas. The term “inert gas” as used in the context of the present invention denotes a gas that is inert with regard to the reaction of N₂O with cyclopentene as well as towards N₂O under the reaction conditions. As inert gases for example nitrogen, carbon dioxide, carbon monoxide, argon, methane, ethane and propane are to be mentioned.

Likewise, gases may be also contained in the mixture (G2) which are not inert gases in the reaction of N₂O with cyclopentene. As such gases, inter alia, NOₓ or, for example, oxygen are to be mentioned. The term “NOₓ” as understood in the context of the present invention refers to all compounds NₐO_{b} besides N₂O, wherein a is 1 or 2 and b is a number from 1 to 6. Instead of the term “NOₓ”, the term “nitrogen oxides” is also used in to the context of the present invention. In such a case, it is preferred to use those mixtures (G2) whose content of these gases is 0.5% by volume at the most, based on the total weight of the mixture (G2).

Accordingly, the present invention also relates to a process as described above, characterized in that the mixture (G2) contains 0.5% by volume of oxygen at the most or at 0.5% by volume of nitrogen oxides the most or at the most both 0.5% by volume of oxygen and 0.5% by volume of nitrogen oxides, each based on the total volume of the mixture (G2). A value of, for example, 0.5% by volume here denotes a total content of all possible nitrogen oxides apart from N₂O of 0.5% by volume.

In principle, the composition of the mixtures in the context of the present invention may be determined by every way known to the skilled person. In the context of the present invention, the composition of the mixture (G2) is preferably determined by gas chromatography. However, it may also be determined by means of UV spectroscopy, IR spectroscopy or wet-chemically.

According to the invention, the mixture (G2) is particularly used in fluid or supercritical form, preferably in supercritical form. In that case according to the invention it is possible to subject the mixture (G2) to a treatment prior to the condensation in order to reduce the concentration of inert and interfering compounds in mixture (G2).

In particular, it is possible in the context of the present invention to use mixtures (G2) which are obtained from industrial scale processes. Should these mixtures (G2) accordingly contain more than 0.5% by volume of oxygen and/or nitrogen oxides, they can generally be used in the process according to the invention. Preferably, these mixtures (G2) as well as such mixtures (G2) of similar composition which are not obtained from industrial scale processes, are subjected to at least one purification step in which the content of oxygen and/or nitrogen oxides is adjusted to at most 0.5% by volume before use in the process according to the invention.

One appropriate mixture (G2) according to the present invention contains preferably 50 to 99.0% by volume of dinitrogen monoxide, 1 to 20% by volume of carbon dioxide and 0 to 25% by volume of other gases. The denoted % by volume each refers to the complete gas mixture (G2). The sum of the single components of the gas mixture (G2) results in 100% by volume.

Preferably, the gas mixture (G2) contains 60 to 95% by volume of dinitrogen monoxide, particularly 70 to 90% by volume, particularly preferably 75 to 89% by volume of dinitrogen monoxide.

The gas mixture can further contain 1 to 20% by volume of carbon dioxide. Preferably, the gas mixture (G2) contains 5 to 15% by volume of carbon dioxide, particularly 6 to 14% by volume of carbon dioxide.

Preferably, the gas mixture (G2) contains 0 to 25% by volume of further gases. The gas mixture G-2 can contain one or more further gases wherein the denoted amount is based on the sum of the contained gases.

Appropriate processes for the preparation of such gas mixtures are generally known to the person skilled in the art.

The residence time of the reaction mixture in the reactor (R1), respectively in the reactors (R1) and (R2) is generally in the range of from 0.1 to 48 h, preferably in the range of from 0.2 to 5 h and particularly preferably in the range of from 0.3 to 2.5 h. Thereby it is conceivable not to keep the temperature or the pressure or both of them constant in the reactor but rather to appropriately vary them in the limits denoted above.

According to the invention, the selectivities for cyclopentanone of the reaction with regard to cyclopentene are for example in the range of from 85 to 99.9%, preferably 90 to 99%, particularly 92 to 97%.

The mixture obtained by the process according to the invention containing cyclopentanone can in principle be further processed in the obtained form. However, according to the invention the obtained mixture can be also worked up according to all appropriate methods for the isolation of cyclopentanone. According to the invention distillative methods are particularly preferably for working up.

In that case the process according to the invention can particularly have a further separation stage that is carried out after the reaction in reactor (R1) or the reaction in reactors (R1) and (R2) or the downstream reactors. The separation stage preferably comprises at least one distillation, yet preferably at least one flash distillation, for example for the separation of N₂ and unreacted N₂O, and one distillation. According to the invention, the distillation can be carried out for example at a pressure from 2 to 6 bar, preferably from 3 to 5 bar, particularly 4 bar and a bottom temperature from 150 to 250° C., preferably from 170 to 200° C., for example 180° C.

According to the invention, preferably at least two flash stages are carried out, for example two, three or four. If for example two flash stages are carried out, these are particularly carried out at a different pressure and a different temperature. Thus, the first flash stage is preferably carried out at a pressure from 15 to 30 bar, preferably from 18 to 26 bar, particularly from 20 to 23 bar and a bottom temperature from 100 to 200° C., preferably from 120 to 180° C., for example from 140 to 160° C. The second flash stage is preferably carried out at a pressure from 1 to 8 bar, preferably from 2 to 6 bar, particularly 3, 4 or 5 bar and a bottom temperature from 50 to 150° C., preferably 75 to 125° C., for example at 85 to 105° C.

According to the invention, the separation stage can comprise at least one flash vessel and one distillation column. After leaving the reactor (R1) or the reactors or after leaving the downstream reactor the reactor discharge is preferably expanded and cooled to separate formed N₂ and unreacted N₂O as gaseous stream. In principle this gaseous stream can be feeded back to the N₂O enrichment, but preferably it is disposed. Preferably, the stream is expanded to a pressure which is slightly above the pressure in the distillation column. The liquid stream is then worked up in one or more distillation columns. Preferably, for example at the top unreacted cyclopentene is separated and, according to the invention, can be feeded back to the reactor at least partially or completely. If necessary, a part of the stream can be discarded to optionally suppress the escalation of by-components. Preferably, these by-components are separated by means of distillation. Substantially cyclopentanone is preferably separated at the bottom.

For example cyclopentanone can be further purified by distillation. In that case the distillation can be carried out according to methods known to the person skilled in the art.

The reactor (R1) used for the process, according to the invention, is also suitable for other reactions, which occur in fluid phase, i.e. in liquid or supercritical phase, wherein at least two educts are reacted in the presence of a homogeneous catalyst or without a catalyst and which are strongly exothermic. Particularly, such reactors are suitable for reactions in which the adiabatic increase in temperature is above 100° C.

Examples for such reactions are in particular the oxidation of olefins, or alkines, respectively, with N₂O, the epoxidation of C—C double bonds with H₂O₂ or hydroperoxides, the oxidation of ketones with nitric acid or the addition of nucleophiles to epoxides, for example of water, alcohols, ammonia, amines, hydroxylamines or hydrazines.

In the following the invention is exemplified in more detail by means of examples.

### EXAMPLES

### Example 1

### Comparative Examples: Reaction in the Tubular Reactor

Example 1 was carried out in a reactor with downstream flash and distillation stage.

The reactor consists of a series of 16 single tubes (outer diameter=10 mm, wall thickness=2 mm, inner diameter=6mm, length=5.3 m) that were spirally coiled (radius r=125 mm, pitch P=30 mm). The reaction volume inclusive the connecting parts is 2510 ml in total.

The tube is equipped with a double jacket (outer tube: outer diameter=20 mm, wall thickness=2 mm, annular gap width=3 mm) through which a cooling liquid is pumped in direct flow to remove the reaction heat (Marlotherm SH of the firm Sasol). The temperature of the entering cooling agent is adjusted by means of an external thermostat at 280±2° C. Directly at the exit of the reactor is a pressure maintenance that keeps the pressure in the reactor constantly at 280 bar.

The fresh cyclopentene is metered into the reactor with 172.5 g/h. Cyclopentene is obtained from the distillation of a C₅-cut of a steam cracker and has the following composition (% by weight): cyclopentene (ca. 93.2%), cyclopentane (ca. 5.7%), 2-methyl-2-butene (ca. 1.1%), 2,2-dimethylbutane (ca. 0.17%).

By mixing of this stream with a cyclopentene feedback stream a stream of the following composition is generated: cyclopentene (ca. 48.8%), cyclopentane (ca. 46.2%), 2-methyl-2-butane (ca. 3.1%), acetone (ca. 2.0%), 2,2-dimethylbutane (ca. 0.96%). This stream is then metered to the reactor by means of a metering pump (rate of flow: 1632 g/h).

As further stream liquid N₂O (content of N₂O>99.5% by volume, firm Messer Griesheim) is metered to the reactor with ca. 99.2 g/h. The molar ratio of cyclopentene to N₂O in the reactor feed is 0.192 mol/mol.

The cyclopentene conversion in a straight passage is 19.6% and the N₂O conversion is ca. 99.6%. After pressure maintenance the reaction discharge is expanded in two stages with two flash vessels being operated at 11 bara and 1.1 bara to a pressure of 1.1 bara and cooled down. The gaseous components are separated and in a secondary cooler (operated at +5° C.) the hydrocarbons contained therein are condensed out as completely as possible. The gas phase (ca. 64.5 g/h) has the following composition: N₂ (96.4% by volume), N₂O (0.41% by volume), ethylene (0.28% by volume), cyclopentene (0.37% by volume), cyclopentane (0.33% by volume), further C₅-components (545 vppm).

The obtained liquid phase is separated in a distillation column (bubble cap column with 5 column units 106 cm*50 mm, each 10 plates). 187.7 g/h of a stream having the following composition (percent by weight) are obtained as bottom product: cyclopentanone (ca. 96.8%), cyclopentane (ca. 1.3%), 4-pentenal (ca. 1.3%), isopropylmethylketone (ca. 0.8%), cyclopentenoxid (ca. 0.5%), cyclopentene-dimers (ca. 0.5%), cyclopentene (ca. 0.03%).

Of the top product containing 43.4% cyclopentene but no cyclopentanone about 14.9 g/h are discarded in order to avoid the increase of by-components, in particular of acetone and 2-methyl-2-butene. The residual (ca. 1460 g/h) is feeded back as cyclopentene-feedback stream to the mixing tap together with fresh-C₅.

According to this operation mode the space-time yield is ca. 72.4 g cyclopentanone/liter/reaction volume/hour. Per kg of cyclopentanone produced ca. 8.2 kg cyclopentene/cyclopentane mixture have to be separated off and for the most part are feeded back. The required energy for the distillation is 0.73 kWh/kg cyclopentanone.

### Example 2

### Reaction in the Micro Reactor

Example 2 was carried out analogous to example 1, wherein the tubular reactor was replaced with a micro reactor and additionally a secondary reactor was used.

The reactor was produced by the firm Heatric (vessel title: Demonstration Mixer-Reactor, Plant item No.: E100, Year built: 2005, Client: BASF, Client P.O. No.: 1086229765/306/D, Serial No,: H1016A, Type: PCR).

The main reactor consists of 48 plates of stainless steel 316/316L with 338*188*1.9 mm in which semi-circular channels were incorporated and that were afterwards diffusion welded to one single block. The finished reactor has the following dimensions: 338*188*91 mm. The two educts are injected through separated inlets and are initially separated in the entry zone (upper plate) and only then they are mixed. The available reaction volume in this main reactor is about 194 ml, spread over 10 plates. In these plates there are two parallel product guiding channels that are semicircular and have a radius of 1.2 mm (cross section area: 2.262 mm²). The channels are arranged parallel on each plate and pass 49 passages on each plate. Thus, the total length of each channel is approximately 45 m.

Cooling liquid is pumped through the cooling circle with ca. 16 l/min to remove reaction heat (Marlotherm SH of the firm Sasol). The temperature of the entering cooling medium is adjusted at 280±2° C. with an external thermostat. Two tube coils arranged in series are used as downstream reactor being also operated at 280±2° C. These tube coils have the same dimensions as those of the reactor used in example 1. The available reaction volume is in total (including connecting parts) ca. 530 mL. At the exit of the last reactor is a pressure maintenance that keeps the pressure in the reactor and in the secondary reactor constantly at 280 bar.

The fresh cyclopentene feed is metered with 205.4 g/h. This one is obtained from the distillation of a C₅-cut of a steam cracker and has the following composition (percent by weight): cyclopentene (ca. 94.7%), cyclopentane (ca. 4.5%), 2-methyl-2-butane (ca. 1.0%), 2,2-dimethylbutane (ca. 0.14%).

By mixing this stream with a cyclopentene feedback stream a stream is generated with the following composition: cyclopentene (ca. 92%), cyclopentane (ca. 6.9%), 2-methyl-2-butane (ca. 1.9%), 2,2-dimethylbutane (ca. 0.32%), acetone (ca. 0.18%).

This stream is then metered to the reactor with a metering pump (entry N₂O, rate of flow: 300 g/h). Liquid N₂O (content of N₂O>99.5% by volume, firm Linde) is separately metered to the reactor with 129.2 g/h. The molar ratio of cyclopentene to N₂O in the reactor feed is ca. 0.72.

The cyclopentene conversion in the straight passage is 65% and the N₂O conversion is ca. 92%. After pressure maintenance the reaction discharge is expanded in two stages with two flash vessels being operated at 11 bara and 1.1 bara to 1.1 bara and cooled down. The gaseous components are separated off and in a secondary cooler the hydrocarbons contained therein are condensed out as completely as possible. The gas phase (ca. 88.8 g/h) has the following composition: N₂ (89.4% by volume), N₂O (7.92% by volume), ethylene (790 vppm), cyclopentene (1.1% by volume), cyclopentane (0.21% by volume), further C₅-components (803 vppm).

The liquid phase is separated in a distillation column (bubble cap column with 5 column units 106 cm*50 mm, each 10 plates). 229 g/l of a stream having the following composition (percent by weight) are obtained as bottom product: cyclopentanone (ca. 92.3%), cyclopentane (ca. 3.1%), 4-pentenal (ca. 1.1%), isopropylmethylketone (ca. 0.34%), cyclopentenoxid (ca. 0.5%), cyclopentene-dimers (ca. 0.25%), cyclopentene (ca. 0.19%).

Of the top product containing 84.8% cyclopentene but no cyclopentanone about 15.4 g/h are discarded in order to avoid the increase of by-components, particularly of acetone and 2-methyl-2-butene. The residual (ca. 95 g/h) is feeded back as cyclopentene-feedback stream to the reactor.

According to this operation mode the space-time yield is ca. 399 g cyclopentanone/liter/reaction volume/hour. Thus, it takes 5.5 times less reaction volume for the same amount of product. Furthermore, per kg produced cyclopentanone only 0.52 kg cyclopentene/cyclopentane mixture have to be separated off and have to be feeded back partially instead of the 8.2 kg according to example 1. The energy required for this is only 0.09 kWh/kg cyclopentanone in stead of the 0.73 kWh/kg according to example 1.

### Example 3

### Reaction in the Microreactor

Example 3 was carried out analogous to example 2, yet without the pipe coils as secondary reactor.

Cooling liquid is pumped in the cooling circuit at a rate of ca. 16 l/min to remove reaction heat (Marlotherm SH of the firm Sasol). The temperature of the entering cooling medium is adjusted to 280±2° C. by use of an external thermostat.

In total the available reaction volume is (including connecting parts) ca. 201 mL. At the exit of the last reactor is a pressure maintenance that keeps the pressure in the reactor constantly at 280 bar.

The fresh cyclopentene feed is metered with 154 g/h. This one is obtained from the distillation of a C₅-cut of a steam cracker and has the following composition (percent by weight): cyclopentene (ca. 94.9%), cyclopentane (ca. 4.2%), 2-methyl-2-butane (ca. 1.0%), 2,2-dimethylbutane (ca. 0.04%).

Initially, this stream is mixed with a cyclopentene-feedback stream to produce a mixture having the following composition: cyclopenten (ca. 93.1%), cyclopentane (ca. 4.3%), 2-methyl-2-butene (ca. 2.3%), 2,2-dimethylbutane (ca. 0.09%), acetone (ca. 0.41%).

This stream is then metered to the reactor with a metering pump (entry N₂, rate of flow 268 g/h).

Liquid N₂O (content of N₂O>99.5% by volume, firm Linde) is separately metered to the reactor with 123 g/h (entry N1). The molar ratio of cyclopentene to N₂O in the reactor feed is ca. 0.72 mol/mol.

The cyclopentene conversion in the straight passage is 54% and the N₂O conversion ca. 76%. After pressure maintenance the reactor discharge is expanded to a pressure of 1.1 bara in two stages with two flash vessels being operated at 11 bara and at 1.1 bara and cooled down. The gaseous components are separated off and in a secondary cooler the hydrocarbons contained therein are condensed out as completely as possible. The gas phase (ca. 61.7 g/h) has the following composition: N₂ (81.7% by volume), N₂O (17.7% by volume), ethylene (674 vppm), cyclopentene (0.4% by volume), cyclopentane (402 ppm), further C₅-components (346 vppm).

The liquid phase is separated in a distillation column (bubble cap column with 5 column units 106*50 mm, in each case 10 plates). 165.4 g/h of a stream with having following composition (% by weight) are obtained as bottom product: cyclopentanone (ca. 93.8%), cyclopentane 3.0%), 4-pentenal (ca. 1.3%), cyclopenteneoxid (ca. 0.5%), cyclopentene-dimers (ca. 0.25%), cyclopentene (ca. 0.9%).

Of the top product containing 91.1% cyclopentene but no cyclopentanone ca. 8.1 g/h are discarded in order to avoid the increase of by-components, in particular acetone and 2-methyl-2-butene. The residual (ca. 114 g/h) is feeded back to the reactor as cyclopentene-feedback stream.

According to this operation mode the space-time yield is ca. 823 g cyclopentanone/liter/reaction volume/hour. Thus, it takes for the same amount of product 11.4 times less reaction volume in comparison to example 1. Furthermore, per kg produced cyclopentanone only 0.11 kg cyclopentene/cyclopentane-mixture have to be separated off and partially feeded back in stead of 8.2 kg according to example 1. The energy required for this is only 0.09 kWh/kg cyclopentanone in stead of 0.73 kWh/kg according to example 1.

1. A process for the preparation of cyclopentanone comprising the reaction of a mixture (G1) containing at least cyclopentene with a mixture (G2) containing at least dinitrogen monoxide, wherein the reaction is carried out in at least one reactor (R1) having channels of a diameter in the range of from 0.1 mm to 50 mm, wherein the reactor has at least two zones (Z1) and (Z2) having channels of a different diameter, and the diameter of the channels of zone (Z1) is smaller than the diameter of the channels of zone (Z2).

2. The process for the preparation of cyclopentanone according to claim 1, wherein the reactor (R1) has at least three zones (Z1), (Z2) and (Z3) having channels of a different diameter, and the diameter of the channels of zone (Z1) is smaller than the diameter of the channels of zone (Z2) and the diameter of the channels of zone (Z2) is smaller than the diameter of the channels of zone (Z3).

3. The process for the preparation of cyclopentanone according to claim 2, wherein the diameter of the channels in zone (Z1) is in the range of from 0.5 mm to 2.0 mm, the diameter of the channels in zone (Z2) is in the range of from 2.5 mm to 6.0 mm and the diameter of the channels in zone (Z3) is in the range of from 6.5 mm to 10.0 mm.

4. The process for the preparation of cyclopentanone according to claim 1, wherein the reaction is carried out in two parallel reactors (R1) and (R2) each having channels of a diameter in the range of from 0.1 mm to 50 mm and, wherein reactor (R1) and reactor (R2) each have at least two zones (Z1) and (Z2) having channels of a different diameter and the diameter of the channels of zone (Z1) each is smaller than the diameter of the channels of zone (Z2).

5. The process for the preparation of cyclopentanone according to claim 1, wherein the mixtures (G1) and (G2) are mixed in the reactor in the channels.

6. The process for the preparation of cyclopentanone according to claim 1, wherein the reaction is carried out at a pressure from 200 to 500 bar and a temperature from 270 to 300° C.

7. The process for the preparation of cyclopentanone according to claim 1, wherein the mixture (G1) contains at least 90% by weight of cyclopentene.

8. The process for the preparation of cyclopentanone according to claim 1, wherein the mixture (G2) contains at least 70% by volume of dinitrogen monoxide.

9. The process for the preparation of cyclopentanone according to any claim 1, wherein the mixture (G2) contains 75 to 99% by volume of dinitrogen monoxide.
